(19) United States
(12) Patent Application Publication
(10) Pub. No.: US 2009/0203295 A1
(43) Pub. Date: Aug. 13, 2009

(54) Brassiere especially for lactating mothers

(76) Inventor: Lee-Anne Kassel, Gauteng (ZA)

Correspondence Address: MERCHANT & GOULD PC, P.O. BOX 2903, MINNEAPOLIS, MN, 55402-0903, US

(21) Appl. No.: 11/916,590

(22) PCT Filed: Jun. 7, 2005
(86) PCT No.: PCT/IB05/01592

§ 371 Date: Aug. 27, 2008

(51) Int. Cl. A41C 3/04; A41C 3/00; A61F 13/15

(52) U.S. Cl. 450/37; 450/86; 604/385.07

(57) ABSTRACT

A brassiere (1) is provided comprising a torso encircling band (2) adapted to encircle the torso immediately beneath the breasts of a wearer and to which is attached a lower edge of a front panel (4) adapted to operatively extend over a pair of breasts. An upper edge (8) of the front panel terminates in a pair of spaced symmetrically located support zones (9) to each of which is attached a supporting shoulder strap (10) such that the torso encircling band and shoulder straps together operatively support the brassiere on a person wearing same. The front panel is made of a fabric that has limited degree of stretch and semi-permeable characteristics in that it is substantially moisture-proof but permeable to vapours and gasses. The lower edge of the front panel has one or more tucks (6) where it attaches to the torso encircling band on each side of a centre thereof so as to define a breast accommodating and supporting pouch (7) on each side of the centre of the panel. A separate inner liner (16) of hydrophobic material has a lower edge attached to the torso encircling band and an upper edge supported with upper edge of the front panel.

### FIELD OF THE INVENTION

This invention relates to a brassiere especially for lactating mothers, and more particularly, to a brassiere that is aimed at preventing moisture from coming into contact with a mother's outer clothing to thereby obviate the formation of wet patches in the outer clothing that can be embarrassing, uncomfortable, and unsightly.

### BACKGROUND TO THE INVENTION

Numerous different brassieres for use by lactating mothers have been proposed and are available on the market. Most, if not all of these, aim at supporting the two breasts in separate, well defined cups each of which may have an opening for providing access to the nipple for a feeding infant. The openings each have a flap for covering the nipple when the breast is not being used for feeding purposes.

Not all prior art brassieres of this type have had such openings and, indeed, in my earlier configuration that is described in international patent application publication number WO 02/080710, such access openings were omitted. In that configuration, the brassiere nevertheless had two well defined cups of generally conventional construction with the distinguishing characteristic that the cups were formed of a permeable fabric such as a suitable knit, for example a suitable Lycra fabric, to the inner face of which was laminated a semi-permeable membrane such as a macro porous polyurethane. The polyurethane membrane is selected to allow vapours and gasses to pass through it but to form a barrier to liquids. An inner layer of lace or netting was provided for comfort purposes on the inside of the cups.

Whilst this configuration provided certain advantages over what was then currently available in the marketplace, it also fails, as in the case of many other configurations, to adequately take account of the fluctuation in size of the breasts from one feed to the next and that is consequently uncomfortable to the wearer, at least from time to time.

### OBJECT OF THE INVENTION

It is, accordingly, an object of this invention to provide a brassiere especially for lactating mothers that provides comfort alongside adequate support whilst also substantially obviating the formation of wet patches in the outer clothing of a wearer.

### SUMMARY OF THE INVENTION

In accordance with this invention there is provided a brassiere comprising a torso encircling band adapted to encircle the torso immediately beneath the breasts of a wearer and support, together with a pair of supporting shoulder straps, a fabric support for the breasts that is of a material having semi permeable characteristics in that it is substantially moisture-proof but permeable to vapours and gasses; the brassiere being characterised in that the fabric support comprises a front panel adapted to operatively extend over a pair of breasts and having a lower edge that is attached to the torso encircling band and an upper edge that terminates in a pair of spaced symmetrically located support zones to each of which is attached a supporting shoulder strap, the front panel being made of a fabric that has a limited degree of stretch and wherein the lower edge of the front panel has one or more tucks where it attaches to the torso encircling band on each side of a centre thereof so as to define a breast accommodating and supporting pouch on each side of the centre of the panel and in that the brassiere has a separate inner liner that has a lower edge attached to the torso encircling band and an upper edge supported with the upper edge of the front panel, the inner layer being made of a woven or nonwoven fabric that is inherently hydrophobic so as to operatively serve as a transfer layer between a wearer's breast and the inside of the front panel whilst itself remaining substantially dry.

Further features of the invention provide for the front panel to be made as a single piece of sheet material; for the front panel to be made of a laminated sheet material having an outer fabric layer and an inner semi-permeable layer that is substantially moisture-proof but permeable to vapours and gasses; for extension straps to have one end thereof secured to the torso encircling band and the other end to an associated shoulder strap thereby limiting the spacing between the support zones and the torso encircling band; and for the two shoulder straps and extension straps to be made of so-called lingerie elastic that inherently has limited stretchability.

Still further features of the invention provide for the brassiere to have rear panel sections that are optionally provided with releasable cooperating fasteners, typically hooks and eyes, in a central position and wherein the rear panel sections are connected to lateral extremities of the front panel at transverse side seams with the upper peripheries of the front panel and rear panels forming a smoothly contoured continuation of each other; for the inner liner to be secured to said side seams at its lateral extremities; and for the upper edge of the inner layer to be unattached to the front panel thereby providing access to a space between them for the optional introduction of one or more absorbent pads therein in which case the inner layer could be stitched to the front panel along lines that define pockets for such absorbent pads.

In order that the invention may be more fully understood one embodiment thereof will now be described with reference to the accompanying drawings.

### BRIEF DESCRIPTION OF THE DRAWINGS

In the drawings:

FIG. 1 is a perspective illustration of one embodiment of brassiere according to the invention in an operative condition from the front lefthand side thereof;

FIG. 2 is a front (from the outside) elevation of the brassiere in an opened out condition; and,

FIG. 3 is a rear (from the inside) elevation of the brassiere illustrated in FIG. 2.

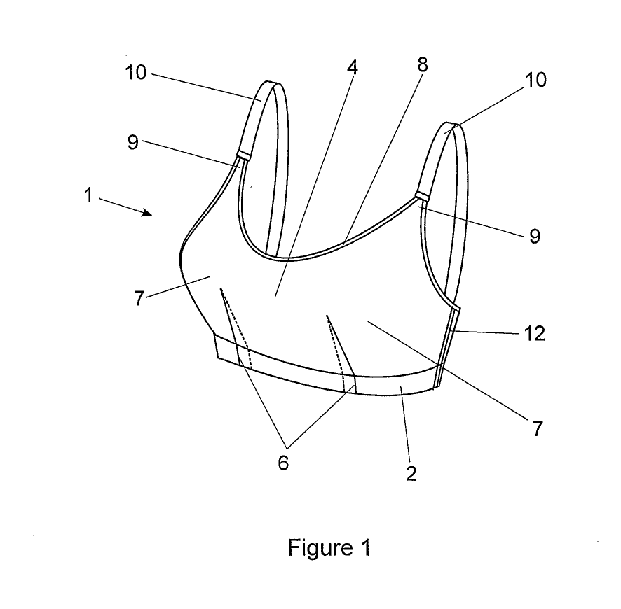

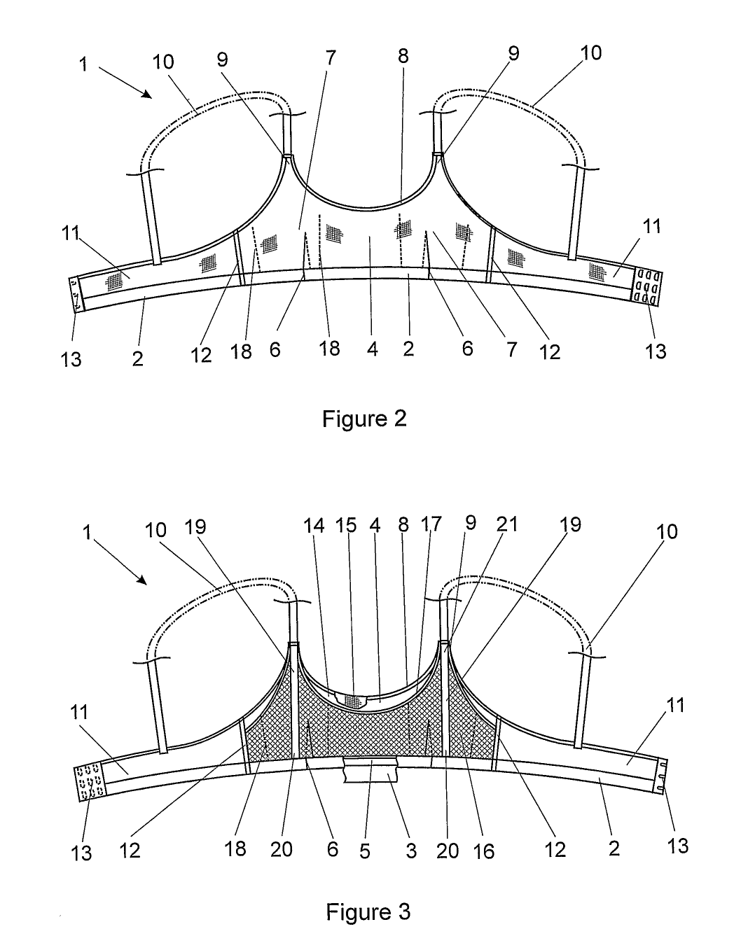

### DETAILED DESCRIPTION WITH REFERENCE TO THE DRAWINGS

In embodiment of the invention illustrated in the drawings a brassiere (1) has a torso encircling band (2) adapted, in use, to encircle the torso of a wearer immediately beneath the breasts in substantially conventional manner. The torso encircling band is made, in this embodiment of the invention, simply by doubling over the operatively lower edge (3) of a front panel (4) with an elastic (5) having limited stretchability enclosed within the resultant seam (see FIG. 3).

At the time of forming this seam, one or more tucks (6) are formed symmetrically on each side of the front panel where it joins the torso encircling band so as to define a breast accommodating and supporting pouch (7) on each side of the centre of the front panel. These tucks naturally form an elongate tapered fold that can be stitched in place so as form what may be more properly termed a dart but the folded material is preferably left unstitched so as to enhance flexibility and versatility. It should be noted that the number and size of the tucks will determine the size of breast accommodating pouch that is formed on each side of the centre and, accordingly, the number and size of tucks will determine different sizes of brassiere.

The front panel is thus adapted to operatively extend over, and support, the pair of breasts of a wearer and the panel has an upper edge (8) that terminates in a pair of laterally spaced, symmetrically located support zones (9) to each of which is attached a supporting shoulder strap (10).

The opposite ends of the shoulder straps are attached to rear panels (11) connected to the torso encircling band and also to the lateral edges of the front panel at side seams (12). The upper periphery of the front panel and that of the rear panels form a smoothly contoured continuation of each other. The free ends of the rear panels are provided with releasable fasteners such as hooks and eyes (13), in substantially conventional manner.

This arrangement is such that the torso encircling band and shoulder straps together operatively support the brassiere on a person wearing same.

The front panel is made of a fabric having a limited degree of stretch and in this embodiment of the invention, is made of a laminated material having an inner semi-permeable membrane layer that is substantially moisture-proof but permeable to vapours and gasses (14) and an outer fabric structural layer (15) (see FIG. 3). Typically the outer layer could be a Lycra containing fabric and the inner layer could be a macro porous polyurethane membrane layer providing the required characteristics.

One type of fabric that has proved to be suitable is a fabric comprising 8% Lycra fibres and 92% cotton fibres and that weighs about 210 grams per square metre. Such a fabric, without the laminated membrane, is already used for certain types of brassieres that are available on the market and is available, for example, from Seaf Fabrics CC of Cape Town South Africa.

A semi-permeable membrane layer that has been found to be particularly suitable is a hydrophilic polyurethane membrane that is available from the company Breathetex (Pty) Ltd of Port Elizabeth, South Africa.

Some of the laminated fabrics described in international patent application publication number WO 01/34080 will apparently also be suitable.

A separate inner liner (16) has a lower edge attached to the torso encircling band; side edges secured to the side seams (12); and an upper edge (17) shaped such that it is supported by the upper edge of the front panel at the support zones. The balance of the upper edge of the liner is located somewhat short of the upper edge of the front panel as shown clearly in FIG. 3.

The inner liner is made of a woven or nonwoven fabric, conveniently a fine mesh type of knitted fabric, that is inherently hydrophobic so as to operatively serve as a transfer layer between a wearer's breast and the inside of the front panel whilst itself remaining substantially dry. It thus serves to “wick” away the moisture and leave the skin rather dry, a most desirable property. One particular material that has been used successfully is a 100 percent polyester knitted (tri cotton 2 bar) running vest or eyelet fabric having a weight of about 48 grams per square metre such as that sold by Torga Linings (Pty) Ltd of Standerton South Africa under the trade reference number 4813405.

The upper edge of the inner layer is preferably unattached to the front panel thereby providing access to a space between them for the optional introduction of one or more absorbent pads therein. If required for such a purpose, the inner layer could be stitched to the front panel along lines (18) to define locating pockets for such absorbent pads.

An extension strap (19) has one end (20) secured to the torso encircling band and the other end (21) secured to an associated shoulder strap (10) towards the lateral side of each of the pouches. The extension strap, in each case, serves to limit the spacing between the associated support zone and the torso encircling band so that the pouch configuration is maintained. Conveniently each of the extension straps is simply an extension of the associated shoulder strap and is positioned substantially in line with the shoulder strap.

The shoulder straps and extension straps are both made of so-called lingerie elastic that inherently has limited stretchability.

It will be understood that a brassiere made as described above clearly overcomes various drawbacks associated with the prior art that is outlined above and that it makes use of certain advantages generally associated only with sports brassieres in a totally new and unexpected application.

It will be appreciated that numerous variations may be made to the embodiment of the invention described above without departing from the scope hereof.

1. A brassiere comprising a torso encircling band adapted to encircle the torso immediately beneath the breasts of a wearer and support, together with a pair of supporting shoulder straps, a fabric support for the breasts that is of a material having semi permeable characteristics in that it is substantially moisture-proof but permeable to vapours and gasses; wherein the fabric support comprises a front panel adapted to operatively extend over a pair of breasts and having a lower edge that is attached to the torso encircling band and an upper edge that terminates in a pair of spaced symmetrically located support zones to each of which is attached a supporting shoulder strap, wherein the front panel is made of a fabric that has a limited degree of stretch and wherein the lower edge of the front panel has one or more tucks where it attaches to the torso encircling band on each side of a centre thereof so as to define a breast accommodating and supporting pouch on each side of the centre of the panel and wherein the brassiere has a separate inner liner that has a lower edge attached to the torso encircling band and an upper edge supported with the upper edge of the front panel, the inner layer being made of a woven or nonwoven fabric that is inherently hydrophobic so as to operatively serve as a transfer layer between a wearer's breast and the inside of the front panel whilst itself remaining substantially dry.

2. A brassiere as claimed in claim 1 in which the front panel is made as a single piece of sheet material.

3. A brassiere as claimed in claim 1 in which the front panel is made of a laminated sheet material having an outer fabric layer and an inner semi permeable layer that is substantially moisture-proof but permeable to vapours and gasses.

4. A brassiere as claimed in claim 1 in which extension straps each have one end thereof secured to the torso encircling band and the other end to an associated shoulder strap thereby limiting the spacing between the support zone and the torso encircling band.

5. A brassiere as claimed in claim 1 in which the two shoulder straps and any extension straps are made of so-called lingerie elastic that inherently has limited stretchability.

6. A brassiere as claimed in claim 1 in which the brassiere has rear panel sections that are provided with releasable cooperating fasteners in a central position and wherein the rear panel sections are connected to lateral extremities of the front panel at transverse side seams with the upper periphery of the front panel and those of the rear panels forming a smoothly contoured continuation of each other.

7. A brassiere as claimed in claim 1 in which the inner liner is secured to said side seams at its lateral extremities.

8. A brassiere as claimed in claim 1 in which the upper edge of the inner layer is unattached to the front panel thereby providing access to a space between them for the optional introduction of one or more absorbent pads therein.

9. A brassiere as claimed in claim 8 in which the inner layer is stitched to the front panel along lines that define pockets for such absorbent pads.

10. A brassiere as claimed in claim 3 in which extension straps each have one end thereof secured to the torso encircling band and the other end to an associated shoulder strap thereby limiting the spacing between the support zone and the torso encircling band.

11. A brassiere as claimed in claim 4 in which the two shoulder straps and any extension straps are made of so-called lingerie elastic that inherently has limited stretchability.

12. A brassiere as claimed in claim 4 in which the brassiere has rear panel sections that are provided with releasable cooperating fasteners in a central position and wherein the rear panel sections are connected to lateral extremities of the front panel at transverse side seams with the upper periphery of the front panel and those of the rear panels forming a smoothly contoured continuation of each other.

13. A brassiere as claimed in claim 5 which the brassiere has rear panel sections that are provided with releasable cooperating fasteners in a central position and wherein the rear panel sections are connected to lateral extremities of the front panel at transverse side seams with the upper periphery of the front panel and those of the rear panels forming a smoothly contoured continuation of each other.

14. A brassiere as claimed in claim 3 which the brassiere has rear panel sections that are provided with releasable cooperating fasteners in a central position and wherein the rear panel sections are connected to lateral extremities of the front panel at transverse side seams with the upper periphery of the front panel and those of the rear panels forming a smoothly contoured continuation of each other.

15. A brassiere as claimed in claim 3 in which the inner liner is secured to said side seams at its lateral extremities.

16. A brassiere as claimed in claim 4 in which the inner liner is secured to said side seams at its lateral extremities.

17. A brassiere as claimed in claim 5 in which the inner liner is secured to said side seams at its lateral extremities.

18. A brassiere as claimed in claim 5 in which the upper edge of the inner layer is unattached to the front panel thereby providing access to a space between them for the optional introduction of one or more absorbent pads therein.

19. A brassiere as claimed in claim 6 in which the upper edge of the inner layer is unattached to the front panel thereby providing access to a space between them for the optional introduction of one or more absorbent pads therein.

20. A brassiere as claimed in claim 7 in which the upper edge of the inner layer is unattached to the front panel thereby providing access to a space between them for the optional introduction of one or more absorbent pads therein.
